(19) United States
(12) Patent Application Publication
(10) Pub. No.: US 2026/0234345 A1
(43) Pub. Date: Aug. 13, 2026

(54) Method for Generating Hydrogel Microparticles

(71) Applicant: Robert Bosch GmbH, Stuttgart (DE)

(72) Inventor: Bernd Scheufele, Koengen (DE)

(21) Appl. No.: 19/469,800

(22) PCT Filed: Mar. 21, 2024
(86) PCT No.: PCT/EP2024/057560

§ 371 Date: Sep. 26, 2025

(30) Foreign Application Priority Data
Mar. 30, 2023 (DE) 10 2023 202 946.4

(51) Int. Cl. C08J 3/075; C12N 5/00

(52) U.S. Cl. CPC C08J 3/075; C12N 5/0018; C08J 2305/04; C08J 2305/12; C12N 2513/00; C12N 2533/90

(57) ABSTRACT

A microfluidic method for the generation of hydrogel microparticles for the cultivation of cells is disclosed. A dispersion medium flows through a second channel. A medium to be dispersed including a hydrogel flows through a first channel and wherein the dispersion medium and the medium to be dispersed meet at a connection point of the first and the second channel. Spherical hydrogel microparticles of the phase to be dispersed are pinched off and conveyed further via a third channel. A hydrophilic culture medium is used as the dispersion medium and a hydrophilic hydrogel cell suspension is used as the medium to be dispersed. A polymerization process of the hydrophilic hydrogel cell suspension is initiated before it comes into contact with the hydrophilic culture medium.

The invention relates to a microfluidic method for generating hydrogel microparticles for cultivating cells according to the preamble of the independent claim.

### PRIOR ART

One area of microfluidics is so-called droplet-based microfluidics. Small droplets or discrete volumes of immiscible liquid phases are generated and used within the microfluidic system. For the microfluidic production of droplets, two liquid phases are used, which are referred to as the continuous phase (medium in which droplets are generated), and the dispersed phase (medium of the droplets). Oils, such as mineral oil or fluorinated oil, are usually used as a continuous phase. Aqueous solutions, such as hydrogels, are used as the dispersed phase.

For cell cultivation, cells may be taken up in the drops and cultivated therein. However, the challenges in cultivation are the sufficient supply of nutrients and oxygen to the cells and ensuring a suitable pH value. Furthermore, the continuous and dispersed phases must be biocompatible and their influences on the cells and cultivation processes must be taken into account.

US 2019/0105279 A1 describes a device for the production of hydrogel microparticles with cells contained therein, wherein a hydrophilic polymer precursor solution with cells contained therein and a hydrophobic liquid, for example, an oil, are brought together at one point through two separate lines, such that hydrogel microparticles are formed. Polymerization of the hydrogel microparticles only takes place after they have formed.

What are referred to as lab-on-a-chip systems (abbreviated as LoC systems) are microfluidic systems that accommodate functionalities of a macroscopic laboratory on a plastic substrate for an automated process. Such systems make it possible to process biochemical processes largely or completely automatically.

Lab-on-a-chip systems typically comprise two main components. The first is a test carrier, for example, in the form of a cartridge, which comprises structures and mechanisms for the manipulation of a received sample, in particular, passive components such as channels, reaction chambers or upstream reagents or also active components such as valves, pumps or mixers. The second main component is a control unit for controlling the microfluidic flows in the cartridge.

### DISCLOSURE OF THE INVENTION

According to the invention, a microfluidic method for generating hydrogel microparticles for cultivating cells is provided with the characterizing features of the independent patent claim.

For example, tumor organoids are used to research tumor diseases, as they represent the conditions in vivo well. One way of producing tumor organoids is to remove individual cells or tissue fragments from a cancer patient's primary tumor and then cultivate them.

According to the invention, a microfluidic method for generating hydrogel microparticles for the cultivation of cells is provided, wherein a dispersion medium, also referred to as continuous phase, flows through a second channel, and wherein a medium to be dispersed comprising a hydrogel, also referred to as dispersed phase, flows through a first channel. The dispersion medium and the medium to be dispersed meet at a connection point of the first and second channels, wherein droplet-shaped or spherical hydrogel microparticles of the phase to be dispersed are pinched off and conveyed further via a third channel. A hydrophilic culture medium is used as the dispersion medium, and a hydrophilic hydrogel cell suspension is used as the medium to be dispersed, wherein a polymerization process of the hydrophilic hydrogel cell suspension is initiated before the hydrophilic culture medium meets the hydrophilic culture medium.

The fact that in the method according to the invention, despite hydrophilic culture medium instead of a hydrophobic liquid, droplets are nevertheless formed when the hydrophilic culture medium and the hydrophilic hydrogel cell suspension are brought together microfluidically, the polymerization process of the hydrogel is initiated before it meets the culture medium, such that there is a significant difference in the rheological properties, in particular, the modulus of elasticity and the viscosity of the two phases. The already slightly polymerized hydrogel cell suspension then meets the aqueous culture medium and the pinch-off of a droplet of the hydrogel cell suspension is made possible.

Instead of a hydrophobic medium, for example, an oil, a hydrophilic culture medium may be used as a dispersion medium or continuous phase. For example, the same culture medium is used which is also used in the established cultivation processes of cells and/or organoids carried out in the laboratory. Particularly advantageous is that an optimal culture medium may be used for cell and/or organoid cultivation. A hydrophobic medium such as an oil, for example, is not an ideal liquid in several respects, as it leads to cytotoxicity and denaturation of proteins on the one hand, and does not serve to supply the cells with nutrients and oxygen like a cultivation medium on the other.

The advantage of using a hydrophilic culture medium as dispersion medium is therefore that it ensures and improves the required supply of nutrients and oxygen to the cells, as well as the setting of a suitable pH value. It is also particularly advantageous that cytotoxic effects on the cells to be cultivated are prevented.

Furthermore, there is improved transferability and comparability between cell and/or organoid cultivation performed manually in the laboratory and the microfluidic implementation of the use of an optimal culture medium for cell and/or organoid cultivation.

For example, the culture medium Dulbecco's Modified Eagle Medium (DMEM) is used as a continuous phase. It is a standardized nutrient medium for cell culture that is established for the cultivation of cells and/or organoids from different tissues or cells.

The cells to be cultivated are suspended in hydrogel or a hydrogel/buffer solution, wherein a suitable cell concentration is settable. In particular, this makes it possible to set the desired number of cells within the generated hydrogel microparticles. The size of the hydrogel microparticles is in the micrometer range and is mainly determined by the flow rates of the continuous and dispersed phase, the interfacial tension between the two phases, and the geometry of the microfluidic channels. For example, it is selected in such a way that one or more cells can be cultivated into organoids therein. The diameters of tumor organoids, for example, are between 100 and 700 μm.

Further advantageous embodiments of the microfluidic device are disclosed in the dependent claims.

In a first advantageous embodiment of the method according to the invention, the polymerization process of the hydrophilic hydrogel cell suspension is thermally induced.

Advantageously, the thermal properties of the hydrogel are utilized. The viscosity and modulus of elasticity of some hydrogels, such as Matrigel® (Corning), agarose or gelatine, are known to be temperature dependent. These two rheological properties and the polymerization process may therefore be selectively controlled by the magnitude and duration of a temperature effect.

It is advantageous if the thermal induction of the polymerization process of the hydrophilic hydrogel cell suspension takes place prior to its introduction into the first channel by tempering the hydrophilic hydrogel cell suspension. If, for example, Matrigel® is used as the hydrogel, the hydrophilic hydrogel cell suspension is tempered to a temperature of 6-10° C., for example. If a different hydrogel or a mixture of various hydrogels is used, the temperature is adjusted to a specific value that initiates the polymerization of the respective hydrogel or mixture.

It is furthermore advantageous if the thermal polymerization process of the hydrophilic hydrogel cell suspension is supported during its flow through the first channel by tempering the first channel.

If Matrigel® is used as a hydrogel, the first channel is tempered to a temperature of 6-10° C., for example.

For this purpose, at least the first channel of the microfluidic device has a temperature control, for example, by means of Peltier elements attached thereto.

Alternatively, it is advantageous if the thermal polymerization process of the hydrophilic hydrogel cell suspension is initiated during its flow through the first channel. The hydrogel cell suspension is then introduced into the first channel of the microfluidic device in a liquid state in which the polymerization process has not yet begun. If Matrigel® (Corning) is used as the hydrogel, the Matrigel cell suspension is, for example, tempered to 4° C. upon introduction into the first channel. The first channel is tempered to initiate the thermal polymerization process of the hydrogel cell suspension. If Matrigel® is used as the hydrogel, the first channel is, for example, tempered to a temperature of 6 to 10° C.

For this purpose, at least the first channel of the microfluidic device has a temperature control, for example, by means of Peltier elements attached thereto. In this way, the polymerization process of the hydrogel is selectively initiated by heat.

Tempering a Matrigel cell suspension and/or the first channel to 6-10° C. is advantageous, as the polymerization process of Matrigel® is thereby initiated and supported in a manner known or verified by experimental investigation. Furthermore, the viscosity values and elastic modulus values of Matrigel® are known for this temperature range, and data on the polymerization duration are available.

Furthermore, it is advantageous if the hydrophilic culture medium is introduced into the second channel pre-tempered. For this purpose, the hydrophilic culture medium is pre-tempered to 37° C., for example, if the dispersing phase comprises Matrigel®. Alternatively or additionally, the second channel is tempered to a temperature of 37° C., for example, using Peltier elements.

The advantage here is that the formation of the spherical hydrogel microparticles is supported when the continuous phase and the phase to be dispersed meet, as well as the complete curing of the hydrogel microparticles during flow through the third channel of the microfluidic device.

In a further advantageous embodiment of the invention, the polymerization process of the hydrophilic hydrogel cell suspension is induced by light. Here, a photoactive hydrogel, for example, polyethylene glycol (PEG), is used. In this embodiment, at least the first channel is optically transparent for the respective wavelengths of light.

The advantage here is that the location or area in which the polymerization process takes place is spatially precisely defined and only takes place where the irradiated light hits the hydrogel cell suspension.

By contrast, in thermal induction, adjacent areas are also tempered, which may possibly influence the polymerization process. Furthermore, it is advantageous that no temperature changes affect the cells or cell cultivation, thereby potentially preventing unwanted or undefined influences.

In a further advantageous embodiment of the invention, the polymerization process of the hydrophilic hydrogel cell suspension is induced by the addition of a reagent.

The reagent is added to the hydrophilic hydrogel cell suspension either before or during the flow through the first channel. If the addition takes place within the microfluidic device during the flow through the first channel, this may be done by a microfluidic mixer or a further supplying microfluidic channel.

An advantage here is that precisely defined material and volume ratios may be set, which allow the polymerization process to proceed in the desired manner. In addition, a plurality of reagents may be mixed or added individually one after another. Compared to the thermal induction of the polymerization process, here too, tempering of adjacent areas and temperature-dependent unwanted effects on the cells and cell cultivation are avoided.

Of course, the above options for initiating the polymerization of the hydrogel cell suspension may be combined with each other in a suitable manner.

This is particularly advantageous when using mixtures of several hydrogels or when using a hydrogel that has more than one crosslinking activation option.

In a particularly advantageous embodiment of the invention, the hydrophilic hydrogel cell suspension has a modulus of elasticity of 0.1-20 Pa and a viscosity of 0.1-50 Pa−s when it meets the culture medium.

In a further particularly advantageous embodiment, the complete polymerization of the hydrogel microparticles occurs during the flow through the third channel by tempering the third channel, in particular to 37° C., and/or by light and/or by a reagent.

Tempering the third channel to a temperature of 37° C. is advantageous with regard to the cultivation of the cells. For 2D and 3D cell cultivation, especially in incubators such as those in laboratories, this is the standard cultivation temperature.

Additionally, Matrigel® and other thermally inducible hydrogels polymerize quickly and completely at 37° C. For this purpose, at least the third channel of the microfluidic device has a temperature control, for example, by means of Peltier elements attached thereto.

For hydrogels whose polymerization process is induced by light, the third channel is irradiated with light for complete polymerization, such that the polymerization process progresses without slowing down or coming to a halt. For this purpose, the third channel is configured to be optically transparent to the respective light wavelengths.

For hydrogels whose polymerization process is reagent-induced, a reagent is, for example, added to the third channel for complete polymerization. The addition takes place, for example, through a microfluidic mixer or another supplying microfluidic channel.

Of course, the above-mentioned options for complete polymerization may be combined with each other in a suitable manner.

It is further advantageously proposed that the hydrophilic hydrogel cell suspension comprises Matrigel® (Corning), agarose, gelatin, and/or PEG and/or alginate.

Matrigel® is preferably used as the hydrogel, which is standardly employed for the cultivation of organoids and whose polymerization or curing is thermally induced. At low temperatures of 4° C., Matrigel® is liquid and no polymerization takes place. The polymerization process initiates at temperatures greater than or equal to 6° C. and is typically carried out in laboratory applications at room temperature or in a laboratory incubator at 37° C. Thus, Matrigel® is proven and easy to handle.

Alternatively or additionally, agarose or gelatine, whose polymerization is thermally induced, is used as a hydrogel. Advantageous when using agarose is that it is non-animal-derived, cost-effective compared to other hydrogels, and readily available.

Another alternative hydrogel used is PEG, for example, whose polymerization is induced by light. Advantageous when using PEG is its synthetic manufacturability and flexibly adjustable mechanical properties.

Another alternative or additional hydrogel used is alginate, for example, which cures by the addition of calcium chloride.

A pumping unit, such as a syringe, membrane, or peristaltic pump, connected to the second microfluidic channel, is used for supplying and advancing the hydrophilic culture medium within the microfluidic device. In particular, this allows the flow rate of the hydrophilic culture medium to be adjusted and varied.

Likewise, a pumping unit, for example, a syringe, diaphragm or peristaltic pump not shown, which is connected to the first microfluidic channel, is used for feeding and advancing the hydrogel cell suspension. This allows the flow rate to be set and varied.

Optionally, an optical monitoring system for the generation of spherical or droplet-shaped hydrogel microparticles, as well as for the number of cells contained therein, may be additionally implemented. For this purpose, optical accessibility of the microfluidic device is to be realized by using at least partially a transparent material in its manufacture. Relevant areas of the microfluidic device may be observed using a camera or microscope unit mounted above it.

In a further step, the cells may be cultivated microfluidically within the generated, fully cured hydrogel microparticles, in particular, to form organoids. For this purpose, the hydrogel microparticles with cells contained therein are transported further, for example, into a microfluidic device as described in the patent application with the file number 102022214275.6, which was filed on Dec. 22, 2022, and used, for example, by means of the method described there for cultivating cells.

The method according to the invention may also be carried out in a cartridge, in particular, a microfluidic cartridge, as described, for example, in DE102016222072A1 or DE102016222075A1.

### BRIEF DESCRIPTION OF THE DRAWINGS

Embodiments of the present invention are illustrated in the drawings and further explained in the subsequent description thereof. The figure shows:

FIG. 1: the schematic representation of a cross-section of a microfluidic device in which the method according to the invention for the production of hydrogel microparticles for the cultivation of cells is carried out, and

FIG. 2: the schematic representation of a flow diagram of an exemplary embodiment of the method according to the invention.

### EMBODIMENTS OF THE INVENTION

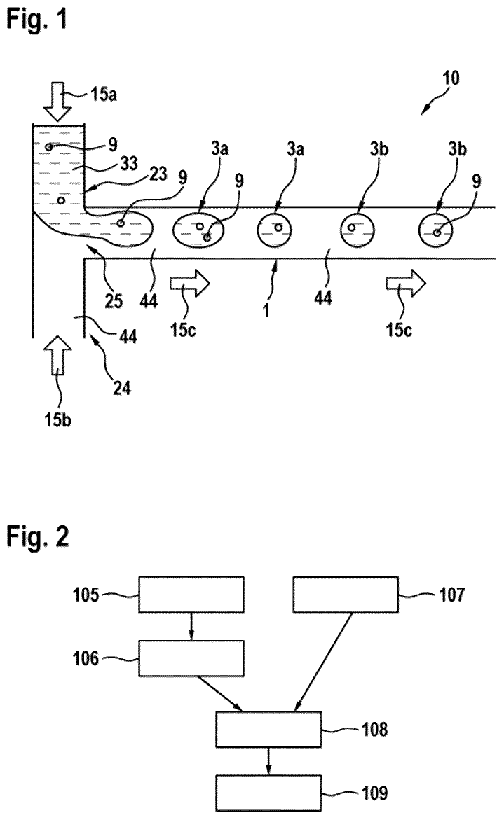

FIG. 1 shows a microfluidic device 10 with a first channel 23 and a second channel 24, which run towards each other and meet at a connection point 25 and then continue in a third channel 1 arranged orthogonally to the first 23 and second channel 24.

In the microfluidic device 10, for example, the method according to the invention for producing hydrogel microparticles 3a, 3b for cultivating cells 9 is carried out. FIG. 1 shows an exemplary embodiment of the microfluidic process in which Matrigel® (Corning), whose polymerization process is thermally induced, is used as the hydrogel of the hydrogel cell suspension 33.

For example, the hydrophilic culture medium 44 DMEM is introduced into the microfluidic device 10 as a dispersion medium 44 via the second channel 24 and conveyed in a second direction 15b. Herein, the hydrophilic culture medium 44 is, for example, preheated to a temperature of 37° C. and/or the second channel 24 is maintained at 37° C. The hydrophilic Matrigel cell suspension 33 is introduced into the microfluidic device 10 as the medium 33 to be dispersed via the first channel 23 and conveyed in a first direction 15a. The hydrophilic Matrigel cell suspension 33 has suspended cells 9 with a set cell concentration and is introduced into the first channel 23 at 4° C. in a liquid state. The first channel 23 is tempered to a temperature of 6-10° C., such that the polymerization process of the Matrigel® is selectively thermally initiated. Thus, when coming into contact with the supplied culture medium 44, a desired modulus of elasticity and a desired viscosity of the Matrigel® are present, which differ significantly from the aqueous culture medium 44. These two rheological properties and the polymerization process are selectively controlled via the magnitude and duration of the temperature effect. Since the Matrigel cell suspension 33 is thus already slightly cured when it meets at the connection point 25 of the first 23 and second channel 24, an already slightly polymerized Matrigel microparticle 3a is pinched off and ultimately torn off and transported onwards. In this way, slightly polymerized droplet-shaped or spherical Matrigel microparticles 3a are dispersed in the culture medium 44 and transported further in a third direction 15c via the third channel 1.

Further polymerization to fully polymerized hydrogel microparticles 3b takes place during the flow through the third channel 1 by tempering the third channel 1 to a temperature of 37° C., for example.

For feeding and advancing the hydrophilic culture medium 44 and the Matrigel cell suspension 33 in the microfluidic device 10, a pumping unit not shown is used, which is connected to the first 23 or second microfluidic channel 24.

The cells 9 in the, in particular, fully cured Matrigel microparticles 3b are then cultivated, for example, within the Matrigel microparticles 3b, in particular, to form organoids.

FIG. 2 shows a flow chart of an exemplary embodiment of the microfluidic process. In a step 105, the hydrophilic hydrogel cell suspension 33 is introduced into the first channel 23 of the microfluidic device 10 as a medium to be dispersed and conveyed further. In a step 106, the polymerization process of the hydrogel is selectively initiated. In a step 107, which takes place, for example, simultaneously with step 105, a hydrophilic culture medium 44 is introduced into the microfluidic device 10 as a dispersion medium via the second channel 24. At the connection point 25 of the first 23 and second channels 24, the partially polymerized hydrophilic hydrogel cell suspension 33 and the hydrophilic culture medium 44 meet, leading to a pinching off of hydrogel microparticles 3a in step 108. These are transported further in step 109 via the third channel 1 and completely polymerized.

1. A microfluidic method for generating hydrogel microparticles for cultivating cells wherein a dispersion medium flows through a second channel, and wherein a medium to be dispersed which comprises a hydrogel flows through a first channel and wherein the dispersion medium and the medium to be dispersed meet at a connection point of the first channel and the second channel, and wherein the hydrogel microparticles of the medium to be dispersed are pinched off, which are conveyed further via a third channel, the microfluidic method comprising:
using a hydrophilic culture medium as the dispersion medium;
using a hydrophilic hydrogel cell suspension as the medium to be dispersed; and
initiating a polymerization process of the hydrophilic hydrogel cell suspension before the hydrophilic hydrogel cell suspension meets the hydrophilic culture medium.

2. The microfluidic method according to claim 1, wherein the polymerization process of the hydrophilic hydrogel cell suspension is thermally induced.

3. The microfluidic method according to claim 2, wherein the thermal induction of the polymerization process of the hydrophilic hydrogel cell suspension is carried out before it is introduced into the first channel by tempering the hydrophilic hydrogel cell suspension.

4. The microfluidic method according to claim 2, wherein the thermal polymerization process of the hydrophilic hydrogel cell suspension is initiated and/or supported during the flow through the first channel by tempering the first channel.

5. The microfluidic method according to claim 1, wherein the hydrophilic culture medium is introduced into the second channel pre-tempered.

6. The microfluidic method according to claim 1, wherein the polymerization process of the hydrophilic hydrogel cell suspension is induced by way of light, and wherein at least the first channel is at least partially optically transparent.

7. The microfluidic method according to claim 1, wherein the polymerization process of the hydrophilic hydrogel cell suspension is induced by adding a reagent which is added to the hydrophilic hydrogel cell suspension before or during the flow through the first channel.

8. The microfluidic method according to claim 1, wherein the complete polymerization of the hydrogel microparticles during the flow through the third channel takes place by tempering the third channel to a temperature of 37° C. and/or by light and/or by a reagent.

9. The microfluidic method according to claim 1, wherein the hydrophilic hydrogel cell suspension has a modulus of elasticity of 0.1-20 Pa and a viscosity of 0.1-50 Pa−s when meeting the hydrophilic culture medium

10. A microfluidic method, wherein the hydrophilic hydrogel cell suspension comprises Matrigel® agarose, gelatin, polyethylene glycol and/or alginate.

11. The microfluidic method according to claim 1, wherein the hydrogel microparticles are spherical hydrogel microparticles.

12. The microfluidic method according to claim 1, wherein the hydrophilic hydrogel cell suspension comprises Matrigel®, agarose, gelatin, polyethylene glycol and/or alginate.

13. The microfluidic method according to claim 2, wherein the thermal induction of the polymerization process of the hydrophilic hydrogel cell suspension is carried out before it is introduced into the first channel by tempering the hydrophilic hydrogel cell suspension to a temperature of 6-10° C.

14. The microfluidic method according to claim 2, wherein the thermal polymerization process of the hydrophilic hydrogel cell suspension is initiated and/or supported during the flow through the first channel by tempering the first channel to a temperature of 6-10° C.

15. The microfluidic method according to claim 1, wherein the hydrophilic culture medium is introduced into the second channel pre-tempered to a temperature of 37° C.
